(19) United States
(12) Patent Application Publication
(10) Pub. No.: US 2014/0046314 A1
(43) Pub. Date: Feb. 13, 2014

(54) ELECTROMAGNETIC THERMOTHERAPY NEEDLE

(71) Applicants: NATIONAL CHENG KUNG UNIVERSITY, Tainan City (TW); METAL INDUSTRIES RESEARCH & DEVELOPMENT CENTRE, Kaohsiung (TW)

(72) Inventors: Cheng-Chi TAI, Tainan City (TW); Shyh-Jier HUANG, Tainan City (TW); Xi-Zhang LIN, Tainan City (TW); Wei-Cheng WANG, Tainan City (TW); Tsung-Chih YU, Tainan City (TW)

(73) Assignees: METAL INDUSTRIES RESEARCH & DEVELOPMENT CENTRE, Kaohsiung (TW); NATIONAL CHENG KUNG UNIVERSITY, Tainan City (TW)

(21) Appl. No.: 13/765,308

(22) Filed: Feb. 12, 2013

(30) Foreign Application Priority Data
Aug. 9, 2012 (TW) 101128781

(51) Int. Cl. A61B 18/18

(52) U.S. Cl. CPC A61B 18/18
USPC 606/33

(57) ABSTRACT

An electromagnetic thermotherapy needle includes a needle body, a covering element, and a holding portion. The material of the needle body includes a magnetically susceptible material. The needle body is formed integrally as one piece and includes a front portion and a rear portion. The front portion has a tip end, and a radial width of the front portion is larger than that of the rear portion. The covering element covers the rear portion, and includes a magnetically non-susceptible material. The holding portion is connected with the covering element or the needle body.

### CROSS REFERENCE TO RELATED APPLICATIONS

This Non-provisional application claims priority under 35 U.S.C. §119(a) on Patent Application No(s). 101128781 filed in Taiwan, Republic of China on Aug. 9, 2012, the entire contents of which are hereby incorporated by reference.

### BACKGROUND OF THE INVENTION

1. Field of Invention

The present invention relates to a thermotherapy needle and, in particular, to an electromagnetic thermotherapy needle.

2. Related Art

The current tumor thermotherapy technologies include microwave thermotherapy, radiofrequency ablation (RFA) therapy, and high-intensity focused ultrasound (HIFU) therapy. Among these therapies, the microwave thermotherapy and RFA therapy are to directly insert a needle electrode into a tumor and then generate heat to cause coagulative necrosis of partial tumor tissue, thereby achieving the purpose of tumor therapy. Herein, the microwave thermotherapy is to generate heat by the microwave generator configured at the tip of the microwave electrode. The RFA therapy is to utilize the radiofrequency current of the electrode needle to excite ions so as to generate heat. However, the electrode needle has complex structure and installing procedure, and can not be used again, which results in high cost. Besides, the electrode needle usually generates very high temperature in the tumor tissues, and the generated heat may be conducted to the peripheral normal tissues. This will cause much pain to the patients.

The HIFU therapy is a technology utilizing the penetrability and focalization of ultrasound wave. The high-intensity ultrasound wave penetrates the skin and then focuses in the deep of the tumor. The focus of the ultrasound wave can generate very high temperature in a short time to directly destroy the tumor. The other tissues located on the path of the ultrasound wave do not accumulate lots of energy, so the normal tissue cells will not be harmed. However, it still causes some side effects, such as partial skin burn or partial pain, during the therapy.

Therefore, it is an important subject of the invention to provide an electromagnetic thermotherapy needle that can decrease the therapy cost and prevent the damage of normal tissues during the therapy, thereby improving the therapy efficiency and product competitiveness.

### SUMMARY OF THE INVENTION

In view of the foregoing subject, and objective of the present invention is to provide an electromagnetic thermotherapy needle that can decrease the therapy cost and prevent the damage of normal tissues during the therapy.

To achieve the above objective, the present invention discloses an electromagnetic thermotherapy needle including a needle body, a covering element, and a holding portion. The material of the needle body includes a magnetically susceptible material. The needle body is formed integrally as one piece and includes a front portion and a rear portion. The front portion has a tip end, and a radial width of the front portion is larger than that of the rear portion. The covering element covers the rear portion, and includes a magnetically non-susceptible material. The holding portion is connected with the covering element or the needle body.

In one embodiment, the magnetically susceptible material comprises metal, alloy, or their combinations. For example, the magnetically susceptible material comprises stainless steel, metal, or glass

In one embodiment, the radial width of the front portion is not smaller than 2.5 times of the radial width of the rear portion, and is not larger than 5 times of the radial width of the rear portion.

In one embodiment, a length of the rear portion is not smaller than 3 times of a length of the front portion, and is not larger than 15 times of the length of the front portion.

In one embodiment, a connection portion of the needle body connecting the front portion and the rear portion is a polygonal rod.

In one embodiment, the tip end has a cone shape or a polygonal tapered shape.

In one embodiment, the magnetically non-susceptible material comprises a ceramic material.

In one embodiment, the covering element covers the rear portion by thermal spraying.

In one embodiment, the material of the holding portion comprises a magnetically non-susceptible material.

As mentioned above, the electromagnetic thermotherapy needle of the present invention includes a needle body containing a magnetically susceptible material. When an alternative magnetic field is applied, the needle body can generate heat for burning or damaging the tumor tissue based on the eddy current effect and/or magnetic hysteresis effect. In addition, the needle body is integrally formed, so that the structural strength thereof can be improved so as to prevent the break of the needle body and thus extend the lifetime thereof. Besides, the tip end of the front portion of the needle body can enhance the penetrability of the needle body. Moreover, since the radial width of the front portion is larger than that of the rear portion, the eddy current and heat generated around the front portion can be increased, thereby improving the burning effect. The covering element covers the rear portion, and includes a magnetically non-susceptible material. Since the magnetically non-susceptible material will not be induced to generate heat by the alternative magnetic field, it is possible to protect the peripheral normal tissues from be harmed by the needle body. The electromagnetic thermotherapy needle of the present invention has simple structure, so that the manufacturing cost and therapy cost can be sufficiently reduced and the therapeutic effect and product competitiveness can be increased.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a schematic diagram showing an electromagnetic thermotherapy needle according to a preferred embodiment of the present invention; and

FIG. 2 is a sectional view of the electromagnetic thermotherapy needle of FIG. 1.

### DETAILED DESCRIPTION OF THE INVENTION

The present invention will be apparent from the following detailed description, which proceeds with reference to the accompanying drawings, wherein the same references relate to the same elements.

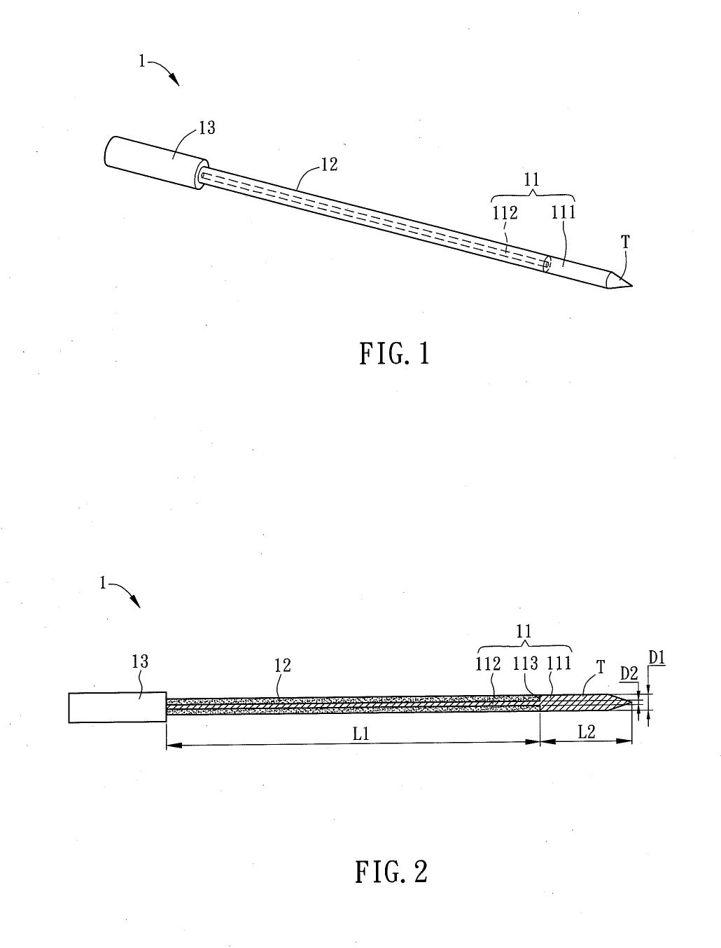

FIG. 1 is a schematic diagram showing an electromagnetic thermotherapy needle 1 according to a preferred embodiment of the present invention, and FIG. 2 is a sectional view of the electromagnetic thermotherapy needle 1 of FIG. 1. The technical features of the electromagnetic thermotherapy needle 1 will be described hereinafter with reference to FIGS. 1 and 2. To be noted, the electromagnetic thermotherapy needle 1 can be applied in cooperating with a high-frequency heater (not shown). The high-frequency heater generates an alternative magnetic field, and the electromagnetic thermotherapy needle 1 is induced to generate an eddy current or heat for burning the targeted tissue.

The electromagnetic thermotherapy needle 1 includes a needle body 11, a covering element 12, and a holding portion 13. The material of the needle body 11 includes a magnetically susceptible material. The magnetically susceptible material comprises metal, alloy, or their combinations. For example, the magnetically susceptible material comprises stainless steel, metal, or glass. The needle body 11 is formed integrally as one piece and includes a front portion 111 and a rear portion 112. The front portion 111 has a tip end T, which has a cone shape or a polygonal tapered shape.

The radial width D1 of the front portion 111 is larger than the radial width D2 of the rear portion 112. This configuration can increase the generated eddy current and heat at the front portion 111 so as to improve the burning effect. Unfortunately, if the radial width D1 is much longer than the radial width D2, the connection between the front portion 111 and the rear portion 112 becomes very weak. Accordingly, the radial width D1 of the front portion 111 is, preferably, not smaller than 2.5 times of the radial width D2 of the rear portion 112, and is not larger than 5 times of the radial width D2 of the rear portion 112. In this embodiment, the radial width D1 is about 1-1.5 mm, and the radial width D2 is about 0.3-0.4 mm.

The front portion 111 is used to stick into the tissue while at least a part of the rear portion 112 stays out of the tissue. If the length of the rear portion 112 is too short, the utility of the needle body 11 will become worse; otherwise, if the length of the rear portion 112 is too long, the entire structural strength of the needle body 11 will be weaker. Preferably, the length L1 of the rear portion 112 is not smaller than 3 times of the length L2 of the front portion 111, and is not larger than 15 times of the length L2 of the front portion 111. In this embodiment, the length L1 is about 10-15 cm, and the length L2 is about 1-3 cm.

In order to enhance the connection strength between the front portion 111 and the rear portion 112, a connection portion 113 of the needle body 11, which connects the front portion 111 and the rear portion 112, is a polygonal rod (e.g. a trapezoid rod). Accordingly, the width of the needle body 11 is gradually decreased from the front portion 111 to the rear portion 112, so that the strength of the needle body 11 can be enhanced.

The covering element 12 covers the rear portion 112, and includes a magnetically non-susceptible material. The magnetically non-susceptible material comprises a non-metal material such as ceramic material or Teflon. Since the magnetically non-susceptible material will not be induced to generate heat by the alternative magnetic field, it is possible to protect the peripheral normal tissues from be harmed by the rear portion 112 of the needle body 11. The covering element 12 can be disposed around the rear portion 112 by many approaches. For example, the covering element 12 may cover the rear portion 112 by thermal spraying. In this embodiment, after covering the rear portion 112, the surface of the covering element 12 is leveled with the surface of the front portion 111. This approach can provide a better appearance and also decrease the resistance while sticking the needle body 11 into the tissue.

The holding portion 13 is connected with the covering element 12 or the needle body 11. In more specific, the holding portion 13 can be directly or indirectly connected with the covering element 12 or the needle body 11. In this embodiment, the holding portion 13 is connected with the covering element 12 and the needle body 11. For example, the rear portion 112 of the needle body 11 is inserted into the holding portion 13, and the covering element 12 is connected to a surface of the holding portion 13. To be noted, the above-mentioned connection method is for illustration only and is not to limit the scope of the present invention. Besides, the material of the holding portion 13 may also comprise a magnetically non-susceptible material, so that it is possible to avoid the heat generated by the holding portion 13.

In summary, the electromagnetic thermotherapy needle of the present invention includes a needle body containing a magnetically susceptible material. When an alternative magnetic field is applied, the needle body can generate heat for burning or damaging the tumor tissue based on the eddy current effect and/or magnetic hysteresis effect. In addition, the needle body is integrally formed, so that the structural strength thereof can be improved so as to prevent the break of the needle body and thus extend the lifetime thereof. Besides, the tip end of the front portion of the needle body can enhance the penetrability of the needle body. Moreover, since the radial width of the front portion is larger than that of the rear portion, the eddy current and heat generated around the front portion can be increased, thereby improving the burning effect. The covering element covers the rear portion, and includes a magnetically non-susceptible material. Since the magnetically non-susceptible material will not be induced to generate heat by the alternative magnetic field, it is possible to protect the peripheral normal tissues from be harmed by the needle body. The electromagnetic thermotherapy needle of the present invention has simple structure, so that the manufacturing cost and therapy cost can be sufficiently reduced and the therapeutic effect and product competitiveness can be increased.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. An electromagnetic thermotherapy needle, comprising:
a needle body formed integrally as one piece and comprising a front portion and a rear portion, wherein the material of the needle body comprises a magnetically susceptible material, the front portion has a tip end, and a radial width of the front portion is larger than that of the rear portion;
a covering element covering the rear portion, wherein the material of the covering element comprises a magnetically non-susceptible material; and
a holding portion connected with the covering element or the needle body.

2. The electromagnetic thermotherapy needle of claim 1, wherein the magnetically susceptible material comprises metal, alloy, or their combinations.

3. The electromagnetic thermotherapy needle of claim 1, wherein the magnetically susceptible material comprises stainless steel, metal, or glass.

4. The electromagnetic thermotherapy needle of claim 1, wherein the radial width of the front portion is not smaller than 2.5 times of the radial width of the rear portion, and is not larger than 5 times of the radial width of the rear portion.

5. The electromagnetic thermotherapy needle of claim 1, wherein a length of the rear portion is not smaller than 3 times of a length of the front portion, and is not larger than 15 times of the length of the front portion.

6. The electromagnetic thermotherapy needle of claim 1, wherein a connection portion of the needle body connecting the front portion and the rear portion is a polygonal rod.

7. The electromagnetic thermotherapy needle of claim 1, wherein the tip end has a cone shape or a polygonal tapered shape.

8. The electromagnetic thermotherapy needle of claim 1, wherein the magnetically non-susceptible material comprises a ceramic material or Teflon.

9. The electromagnetic thermotherapy needle of claim 1, wherein the covering element covers the rear portion by thermal spraying.

10. The electromagnetic thermotherapy needle of claim 1, wherein the material of the holding portion comprises a magnetically non-susceptible material.
